(19) United States
(12) Patent Application Publication
(10) Pub. No.: US 2003/0236505 A1
(43) Pub. Date: Dec. 25, 2003

(54) Cannula

(76) Inventors: Frank Bonadio, Bray (IE); Alan Reid, Dublin (IE); Derek Young, Blackrock (IE); John Butler, Blackrock (IE); Frank Harewood, Kingston (IE)

Correspondence Address: Finnegan, Henderson, Farabow,, Garrett & Dunner, L.L.P., 1300 I Street, N.W., Washington, DC, 20005-3315, US

(21) Appl. No.: 10/346,059

(22) Filed: Jan. 17, 2003

Related U.S. Application Data
(63) Continuation of application No. PCT/IE01/00093, filed on Jul. 23, 2001.

(30) Foreign Application Priority Data
Jul. 21, 2000 (IE) 2000/0590
Dec. 21, 2000 (IE) 2000/1071

(51) Int. Cl. A61M 25/00

(52) U.S. Cl. 604/264; 604/526

(57) ABSTRACT

A cannula (1) comprises a shaft (2) extending between a proximal end (3) located externally of an operating space (4) and a distal end (5) inserted into the operating space (4). At least portion of the shaft (2) is malleable to maintain the distal end (5) of the shaft (2) in a desired manipulated position and/or orientation within the operating space (4) for insertion of a surgical instrument (10) through the cannula (1) for carrying out a surgical procedure within the operating space (4).

This invention relates to cannulae and in particular to cannulae suitable for laparoscopic surgery, such as hand-assisted laparoscopy.

Conventional open surgery requires the creation of an incision in the abdominal wall to allow access to, and visualisation of internal organs and other anatomical structures. These incisions must be large enough to accommodate a surgeon's hands and any instruments to be utilised by the surgeon during the surgery. Traditionally the size of these incisions has been dictated by the need to see, retract and palpate internal bodily structures. While a large incision will provide access to the interior of the abdomen, such incisions are associated with long healing times, are susceptible to infection, and result in unsightly scars.

Laparoscopic surgery is an alternative to open surgery. In this method of surgery, the surgeon operates through small incisions using remotely actuated instruments passed through the abdominal wall using a device called a cannula which creates a working channel. These working channels typically have a radial dimension in the range of from 2 to 15 millimetres. Vision is provided using a laparoscope which is typically 20 to 25 centimetres long and uses fibre-optic technology or a CCD camera to provide the operator with a picture of the interior of the abdomen. The abdomen is generally insufflated with a gas such as carbon dioxide or nitrogen to create a bubble effect and to provide a viable working space, known as the pneumoperitoneum, in which the operator may perform the surgery. Cannulae through which instruments are inserted are constructed to prevent loss of the insufflation gas through them, which would otherwise result in collapse of the pneumoperitoneum.

The benefits of laparoscopic surgery are numerous. Recovery times have been shown to be reduced due to the absence of a large incision. This has benefits for the patient, the health care organisation and society. The benefits to the patient are reduced stay in hospital, faster mobilisation and return to normal activity. The benefits to the health care organisation are also due to the reduced stay in hospital which is often the most expensive aspect of health care provision. Society benefits in faster return to work and normal activity of the patient.

Because of technical difficulties in performing laparoscopic surgery its general indications exist largely in simple surgeries such as lap choli, more advanced laparoscopic surgery requires the use of small incisions and is not the conventional approach.

Laparoscopic surgical techniques are generally complex and surgeons tend to require long periods training to master these techniques. The surgeon manipulates organs and carries out delicate tasks using remotely actuated instruments. Because the surgeon is insulated from the material that he is working on, tactile feedback and the ability to palpate delicate structures is not possible.

The image viewed by the surgeon is a two dimensional image on a video screen, without three dimensional perspective of depth, and distance, and awareness of the proximity of other structures.

Recently, new surgical techniques have been developed that combine the advantages of both open surgery and laparoscopic surgery. In these new techniques, surgery is carried out using a laparoscopic approach with an additional, slightly larger incision to allow the surgeon to insert a hand into the insufflated abdomen. This is often referred to as hand-assisted laparoscopic surgery (HALS).

HALS allows surgeons to retain tactile feedback and three-dimensional perspective. It also permits rapid finger dissection, enhanced retraction capabilities and simplified haemostasis. There are several publications in the literature describing procedures carried out using a hand-assisted approach. These include total and sub-total colectomy, rectopexy, Nissen's fundoplication, gastrectomy, splenectomy, nephrectomy, pancreatectomy and others. Some of these procedures were previously performed using an open technique only.

During laparoscopy or HALS, a laparoscopic instrument is passed into an operating space through a laparoscopic cannula. The instrument is then moved into a desired position in the operating space. It is frequently desirable to approach an organ or piece of tissue in the operating space with the instrument in a particular desired orientation.

However, conventional laparoscopic cannulae are difficult to manipulate as it is only possible to translate the cannula axially through the opening to the operating space, or to pivot the cannula about the opening. Because the instrument is passed through the cannula, manipulation of the instrument is accordingly also difficult.

In this way, the incision point restricts the degrees of freedom of the cannula and/or the instrument, and make it difficult to approach an anatomical structure in a desired manner within the operating space. The restricted freedom of movement may require the surgeon to assume an uncomfortable position during the procedure leading to surgeon fatigue, and extended time periods to complete a procedure.

To access a desired position in the operating space and/or at a desired orientation in the operating space with the instrument, it may be necessary to create another opening to the operating space and to pass the instrument through a cannula at this other opening into the operating space. This causes further trauma to the patient.

Floppy cannulae are also known. For example, U.S. Pat. No. 5,383,861 discloses a flexible cannula for use in endoscopic applications. Such floppy cannulae restrict the surgeon's freedom of movement as the surgeon must hold the floppy cannula in a desired position and/or at a desired orientation in the operating space throughout the laparoscopic procedure.

There is therefore a need for a cannula which will provide an accessway for an instrument through an opening into an operating space without restricting the freedom of movement of a surgeon.

### STATEMENTS OF THE INVENTION

According to the invention there is provided a cannula comprising a shaft extending between a proximal end for location externally of an operating space and a distal end for insertion into the operating space, at least portion of the shaft being malleable to maintain the distal end of the shaft in a manipulated position and/or orientation.

In one embodiment of the invention the shaft is malleable substantially along the length thereof.

In another embodiment the shaft has a distal portion adjacent the distal end of the shaft, and the distal portion of the shaft is malleable. Preferably the shaft has an intermediate portion intermediate the distal end of the shaft and the proximal end of the shaft, and the intermediate portion of the shaft is rigid.

In a further embodiment the shaft has an intermediate portion intermediate the distal end of the shaft and the proximal end of the shaft, and the intermediate portion of the shaft is malleable. Preferably the shaft has a distal portion adjacent the distal end of the shaft, and the distal portion of the shaft is rigid. Alternatively the shaft may have a distal portion adjacent the distal end of the shaft, and the distal portion of the shaft is flexible.

Ideally the distal portion of the shaft is short relative to the intermediate portion of the shaft.

The malleability of the malleable portion of the shaft may vary along the length of the malleable portion of the shaft.

In a preferred embodiment the shaft comprises a main shaft body and a shaft extension mounted to the main shaft body. Ideally the shaft extension is releasably mounted to the main shaft body. Most preferably the shaft extension is mounted to a distal end of the main shaft body.

In one case at least portion of the shaft is extendable from a retracted configuration to an extended configuration. Ideally the shaft is telescopically extendable. Alternatively the shaft may be extendable in a concertina manner.

Desirably the cannula comprises an adjustor to facilitate manipulation of the position and/or orientation of the distal end of the shaft from the proximal end of the shaft.

In a preferred embodiment the cannula comprises a lock for locking the distal end of the shaft in a desired manipulated position and/or orientation.

In either case the adjuster and/or the lock may be provided by at least one wire extending along at least portion of the shaft. Ideally the wire is at least partially malleable. Preferably the wire is slidably received in at least one guide on the shaft. Most preferably the wire comprises a stop to limit movement of the wire.

In a preferred embodiment of the invention at least the malleable portion of the shaft is of a layered construction. At least one layer may comprise a seal. Preferably at least one layer comprises a spring coil. In one case at least one layer is of a polymeric material. Ideally the polymeric material is polyvinylchloride. In another case at least one layer is of a metallic material. Preferably the metallic material is aluminium.

In one case the radial dimension of the shaft is in the range of from 2 mm to 15 mm.

The cannula is preferably a laparoscopic cannula.

In another aspect the invention provides a surgical apparatus comprising a cannula of the invention and a surgical instrument for partial insertion through the cannula.

Preferably the instrument has a distal portion adjacent a distal end of the instrument, and at leas, the distal portion of the instrument is flexible.

According to a further aspect, the invention provides a method of performing minimally invasive surgery, the method comprising the steps of:—

providing a cannula, the cannula having a proximal end and a distal end, and at least portion of the cannula being malleable;

partially inserting the cannula through an opening into an operating space so that the proximal end is located externally of the operating space and the distal end is located within the operating space; and

manipulating the distal end of the cannula into a desired position and/or orientation, the manipulated position and/or orientation being maintained by the malleable nature of the cannula.

Ideally the cannula is at least partially manipulated before insertion of the cannula into the operating space.

The cannula may be at least partially manipulated during insertion of the cannula into the operating space. Preferably the cannula is at least partially manipulated by levering the proximal end of the cannula about the opening to the operating space when the cannula is partially inserted through the opening to the operating space.

The cannula may be at least partially manipulated after insertion of the cannula into the operating space.

In a preferred embodiment the method comprises the steps of:—

providing a surgical instrument; and

partially inserting the surgical instrument through the cannula into the operating space.

Ideally the surgical instrument is inserted through the cannula after the distal end of the cannula has been manipulated into the desired position and/or orientation.

Desirably the method comprises the step of inserting a hand into the operating space. Most preferably the method comprises the steps of:—

providing a hand-access device;

mounting the hand-access device at an opening to the operating space; and

inserting the hand through the hand access device into the operating space.

The cannula may be at least partially manipulated into the desired position and/or orientation by the hand from within the operating space.

The cannula may be at least partially manipulated into the desired position and/or orientation from the proximal end of the cannula externally of the operating space.

In one embodiment the method comprises the step of locking the distal end of the cannula in the manipulated position and/or orientation.

The invention provides in one case a method of performing minimally invasive abdominal surgery wherein the operating space is an abdominal cavity.

The method preferably comprises the step of extending the cannula from a retracted configuration to an extended configuration.

The method may comprise the step of mounting an extension to the cannula.

According to a further aspect of the invention, there is provided a cannula comprising a shaft extending between a proxial end for location externally of an operating space and a distal end for insertion into the operating space, at least portion of the shaft being flexible to manipulate the shaft into a desired position and/or orientation, and a lock for locking the shaft in the manipulated position and/or orientation.

In yet another aspect, the invention provides a cannula comprising a shaft extending between a proximal end for location externally of an operating space and a distal end for insertion into the operating space, at least portion of the shaft being non-straight to access a desired position and/or at a desired orientation within the operating space.

The cannula of the invention is at least partially malleable. The malleability of the cannula enables the cannula to be inserted through an opening into an operating space in a low-profile, substantially straightened configuration, and then enables the cannula to be easily manipulated into a desired position and/or orientation within the operating space. Most importantly, the cannula maintains this position without requiring assistance from separate means to hold the cannula in the desired manipulated position and/or orientation. A device, such as a laparoscopic instrument, may then be passed through the cannula to exit the distal end of the cannula in a desired position and/or at a desired orientation within the operating space.

The cannula of the invention is particularly applicable to hand assisted surgery and in particular to surgical techniques in which an opening is formed in the abdomen, a sealing hand access device is placed in the opening and a surgeon's hand is then inserted through the hand access device into the operating space for carrying out procedures in the operating space. One such sealing device is described in our International patent application published under number WO-A-00/32117, the entire contents of which are incorporated herein by reference. The sealing device seals to the wound edge and to a surgeon's arm to maintain pneumoperitoneum in the operating space.

In this case a cannula is inserted into the operating space through another opening and the position and/or orientation of the distal end of the cannula is controlled by the surgeon's hand. A surgical instrument is then inserted through the cannula and guided either externally or internally by the surgeon's hand to carry out a desired procedure.

It will be appreciated that another laparoscopic instrument may be used alternatively or additionally to the surgeon's hand to manipulate the cannula.

### BRIEF DESCRIPTION OF THE DRAWINGS

The invention will be more clearly understood from the following description of some embodiments thereof, given by way of example only, with reference to the accompanying drawings, in which:—

FIGS. 1 and 2 are schematic views illustrating the introduction of a cannula according to the invention through an opening into an operating space;

FIG. 3 is a schematic view illustrating manipulation of the cannula of FIGS. 1 and 2 within the operating space;

FIG. 4 is a schematic view of the cannula of FIG. 3 after manipulation;

FIG. 5 is a schematic view illustrating partial insertion of an instrument through the manipulated cannula of FIG. 4;

FIG. 6 is a schematic view illustrating partial insertion of the cannula of FIGS. 1 to 5 through the opening into the operating space after manipulation;

FIGS. 7 and 8 are schematic views illustrating manipulation of the cannula of FIGS. 1 to 5 during partial insertion through the opening into the operating space;

FIG. 9 is a schematic view of another cannula according to the invention partially inserted through the opening to the operating space;

FIG. 10 is a perspective view of part of the cannula of FIG. 9;

FIG. 11 is a schematic view illustrating manipulation of the cannula of FIG. 9 within the operating space;

FIG. 12 is a schematic view illustrating locking of the manipulated cannula of FIG. 11 in a desired position and/or orientation;

FIGS. 13 to 15 are schematic views illustrating manipulation of another cannula according to the invention within the operating space;

FIGS. 16 to 18 are schematic views of another cannula according to the invention, similar to FIGS. 2 to 4;

FIG. 19 is a schematic view illustrating mounting of an extension to another cannula according to the invention;

FIG. 20 is a schematic view illustrating manipulation of the cannula of FIG. 19 within the operating space;

FIG. 21 is a schematic view illustrating partial insertion of an instrument through the manipulated cannula of FIG. 20;

FIG. 22 is a schematic view of another cannula according to the invention partially inserted through the opening into the operating space;

FIG. 23 is a schematic view illustrating partial insertion of an instrument through the extended cannula of FIG. 22;

FIG. 24 is a schematic view illustrating partial insertion of an endoscope through the manipulated cannula of FIG. 20;

FIG. 25 is a side, partially cross-sectional view of a shaft of a cannula according to the invention;

FIG. 26 is cross sectional view of the shaft of FIG. 25 on the line A-A;

FIGS. 27 and 28 are views similar to FIGS. 25 and 26 illustrating formation of the shaft of FIGS. 25 and 26; and

FIGS. 29 and 30 are views similar to FIGS. 25 and 26 of a shaft of another cannula according to the invention.

### DETAILED DESCRIPTION

In this specification, the term “malleable” is used to denote an element which is capable of being manipulated into a desired position and/or orientation, and which retains this manipulated position and/or orientation under the typical stresses and strains applied when used for an intended purpose with a patient, for example during partial insertion of a laparoscopic instrument through a malleable cannula.

In this specification, the term “flexible” is used to denote an element which is capable of being manipulated into a desired position and/or orientation, but which does not retain this manipulated position and/or orientation without the assistance of a separate means to hold the flexible element in the manipulated position and/or orientation.

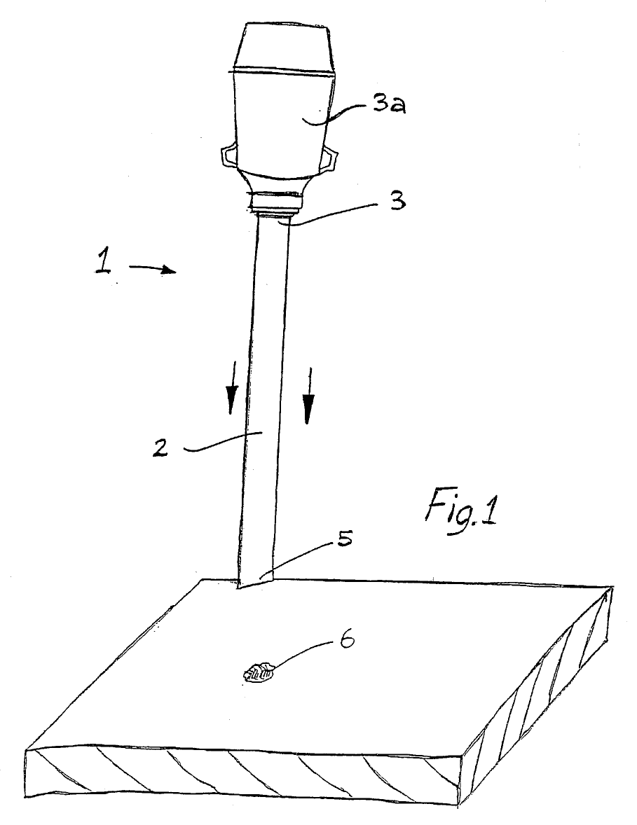

Referring initially to FIGS. 1 to 5, there is illustrated a cannula 1 according to the invention. The cannula 1 comprises a shaft 2 extending between a proximal end 3, which in use is located externally of an operating space 4, and a distal end 5, which in use is inserted into the operating space 4. The proximal end 3 has a conventional instrument entry port 3a incorporating a lip seal.

The cannula 1 is particularly suitable for use with a small opening 6 to the operating space 4, such as a trocar puncture opening as typically used during laparoscopic surgery.

An example of use of the cannula 1 of the invention is to create an access way to an abdominal cavity during a minimally invasive abdominal surgical procedure.

In the cannula 1 of the invention, at least portion of the shaft 2 is malleable. In this case, the shaft 2 is malleable along substantially the entire length thereof.

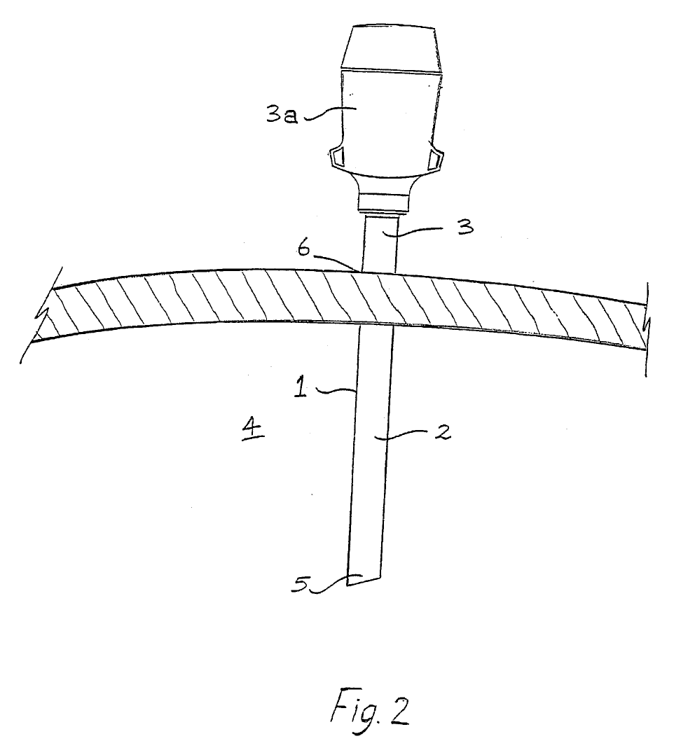

The malleable nature of the shaft 2 enables the shaft 2 to be partially inserted through the opening 6 to the operating space 4 in a low-profile, substantially straightened configuration, as illustrated in FIGS. 1 and 2, while allowing the shaft 2 to be manipulated and allowing the distal end 5 of the shaft 2 to maintain a desired manipulated position and/or orientation without requiring assistance from a separate means to hold the shaft 2 in the manipulated position and/or orientation.

In use, the cannula 1 is inserted partially through the opening 6 to the operating space 4 so that the proximal end 3 of the shaft 2 is located externally of the operating space 4, and the distal end 5 of the shaft 2 is located within the operating space 4 (FIGS. 1 and 2).

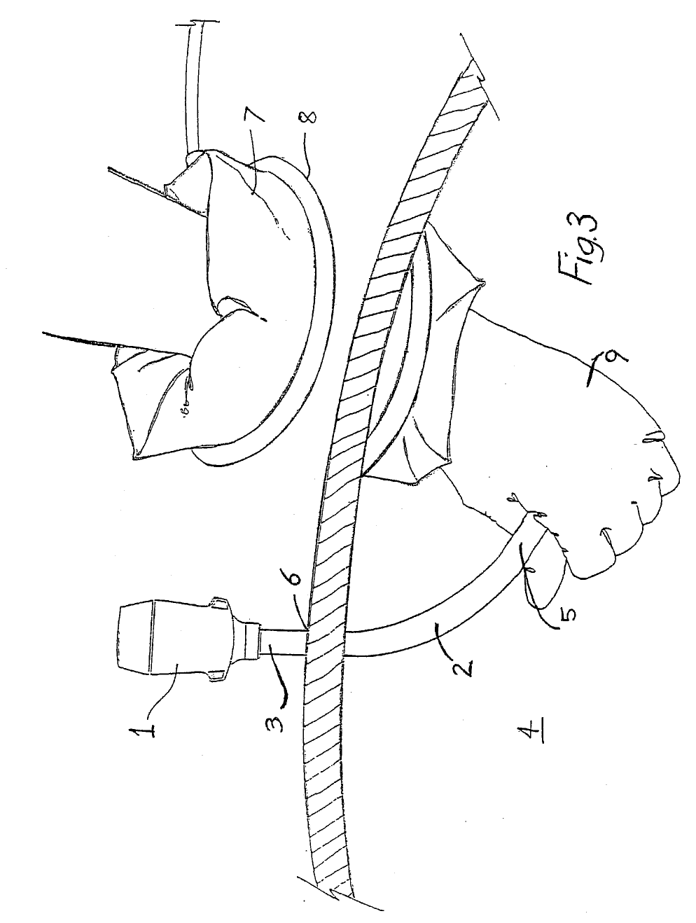

A hand-access device 7 is mounted at another opening 8 to the operating space 4, and a surgeon's hand 9 is inserted through the hand-access device 7 into the operating space 4. The hand-access device 7 is similar to that described in our International patent application published order number WO-A-00/32117, the entire contents of which are incorporated herein by reference. The shaft 2 is manipulated by the hand 9 within the operating space 4 until the distal end 5 is in a desired position and/or orientation within the operating space 4 (FIG. 3).

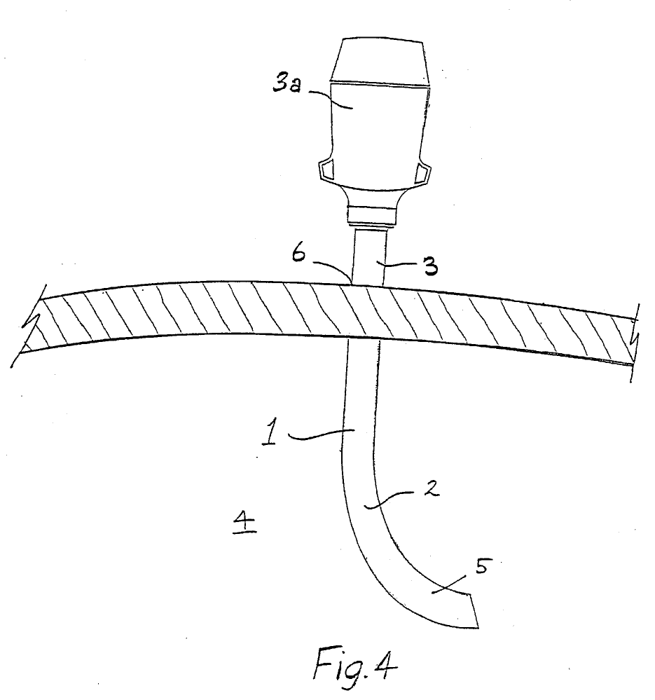

The malleable nature of the shaft 2 ensures that the distal end 5 maintains its manipulated position and/or orientation within the operating space 4, even after the shaft 2 has been released by the hand 9 (FIG. 4).

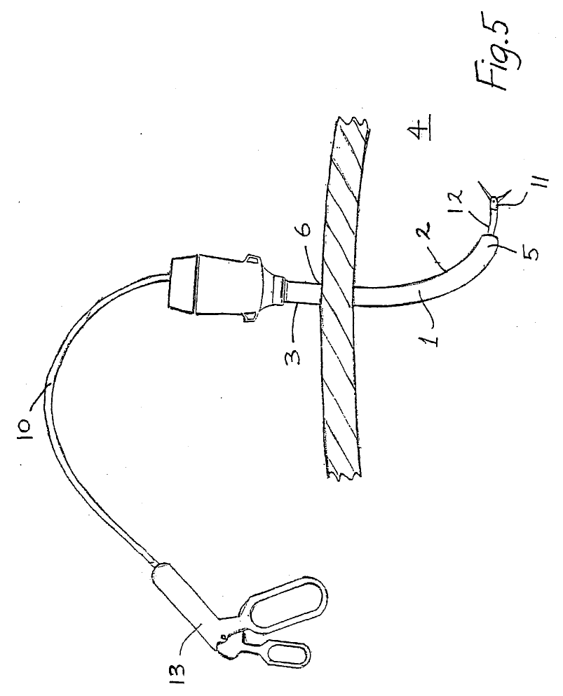

A surgical instrument 10 may then be partially inserted through the cannula 1 into the operating space 4. The malleable shaft 2 ensures that au end effector 11 at a distal end 12 of the instrument 10 exits from the manipulated distal end 5 of the shaft 2 at the desired position and/or orientation in the operating space 4 (FIG. 5).

The cannula 1 of the invention is particularly suitable for positioning and/or orienting the end effector 11 of the instrument 10 when the instrument 10 is flexible. An actuating handle 13 of the flexible instrument 10 may be held in any desired position and/or orientation by a user externally of the operating space 4, as illustrated in FIG. 5. In this manner, the flexible instrument 10 provides the user with enhanced freedom to operate the instrument 10.

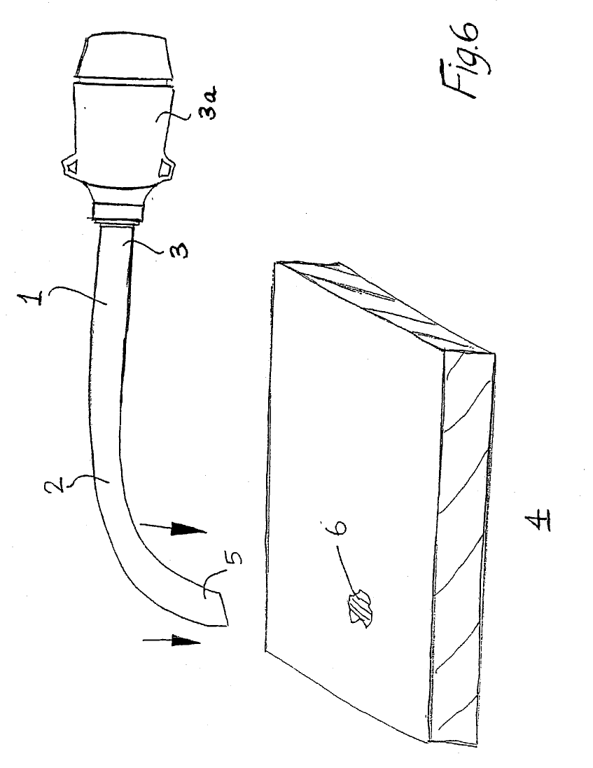

Referring to FIG. 6 there is illustrated an alternative manipulation of the cannula 1. In this case, the shaft 2 is manipulated by the surgeon's hand 9 into a “goose-neck” configuration externally of the operating space 4, and the manipulated shaft 2 is then partially inserted through the opening 6 to the operating space 4 until the distal end 5 of the shaft 2 is in a desired position and/or orientation within the operating space 4. It will be appreciated that the manipulated shaft 2 of FIG. 6 should be gradually rotated during the insertion of the shaft 2 partially through the opening 6 to accommodate the curved shaft 2.

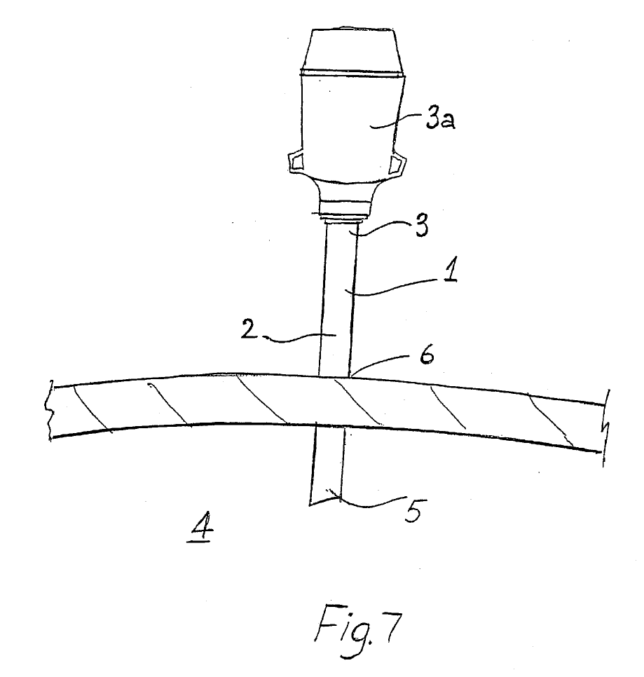

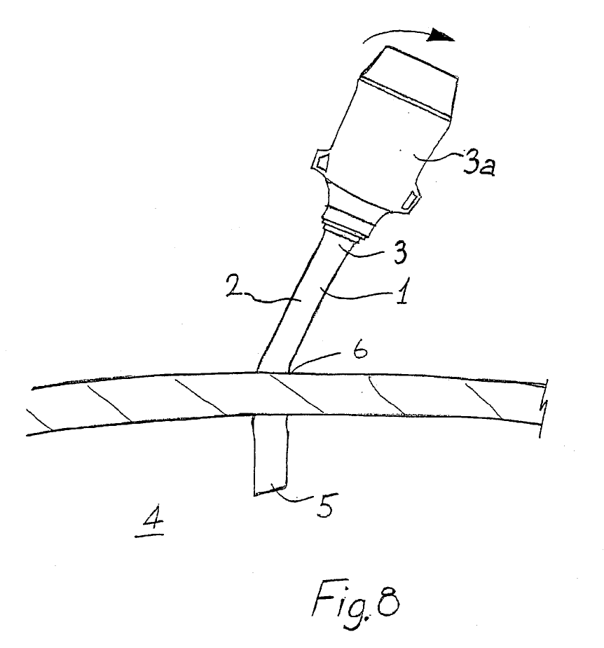

FIGS. 7 and 8 illustrate another alternative manipulation of the cannula 1. In this case, the shaft 2 is manipulated by levering the proximal end 3 of the shaft 2 about the opening 6 to the operating space 4 when the shaft 2 is inserted only partially through the opening 6. The manipulated shaft 2 is then inserted further through the opening 6 until the distal end 5 of the shaft 2 is at a desired position and/or orientation within the operating space 4.

The malleable nature of the shaft 2 enables the manipulation of the shaft 2 to be performed during the insertion stage. This is a highly advantageous aspect of the invention as it allows a user to both insert and manipulate the shaft 2 using only one hand. Use of a separate hand access port is not necessary in this case.

Depending on the physiological characteristics of the patient, for example the thickness of the abdomen or the strength of the abdomen muscle, it may be highly desirable to manipulate the distal end 5 of the shaft 2 when the shaft 2 is partially inserted using the abdomen wall as a fulcrum. By inserting the shaft 2 in a manipulated configuration the surgeon may subsequently gain access with the surgical instrument 10 to regions of the operating space 4 which are laterally remote of the wound opening 6.

It will be appreciated that the manipulation of the shaft 2 may be performed before insertion, during insertion, or after insertion of the shaft 2 partially through the opening 6 to the operating space 4, or by any suitable combination of these manipulations.

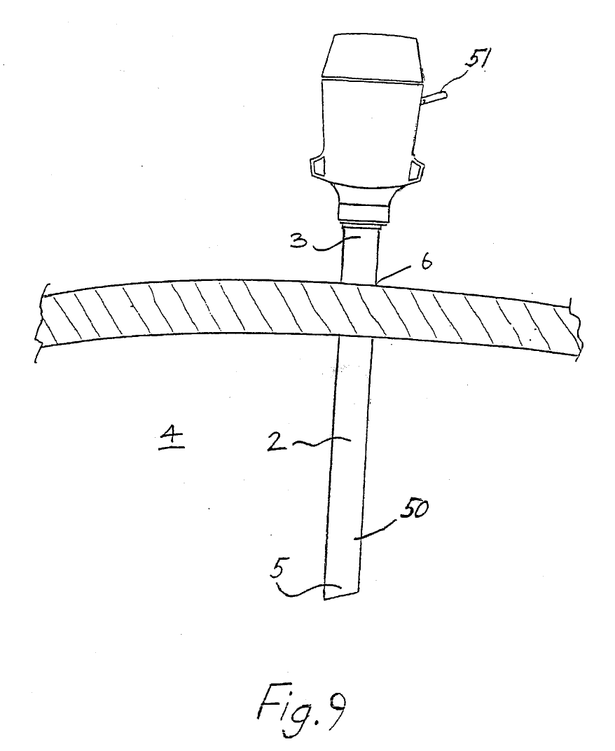

Referring now to FIGS. 9 to 12 there is illustrated another cannula 50 according to the invention, which is similar to the cannula 1 of FIGS. 1 to 8, and similar elements in FIGS. 9 to 12 are assigned the same reference numerals.

In this case, the cannula 50 comprises means to facilitate locking of the distal end 5 of the shaft 2 in a desired manipulated position and/or orientation.

The locking means ensures that the distal end 5 of the malleable shaft 2 maintains its desired manipulated position and/or orientation within the operating space 4, even if the shaft 2 is inadvertently knocked against by the surgeon's hand 9, or by a laparoscopic instrument, or by an internal organ. The locking means also ensures that the malleable shaft 2 maintains the desired position and/or orientation during insertion of the laparoscopic instrument 10 through the cannula 50. This is particularly advantageous if the instrument 10 is not completely flexible.

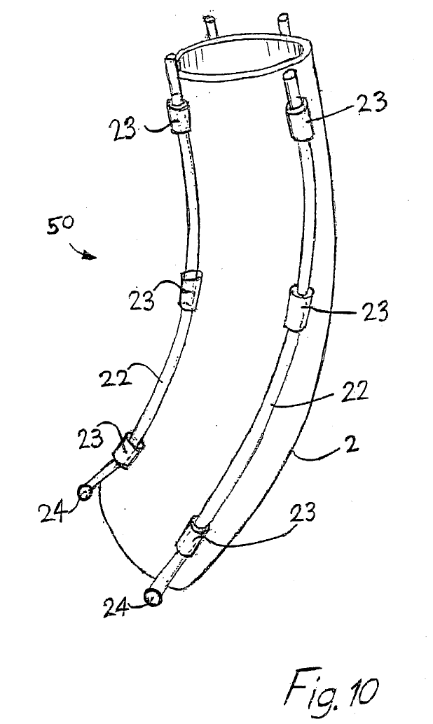

The locking means comprises at least one malleable wire 22, extending along at least portion of the shaft 2, the wire 22 being slidably received in co-operating guides 23 on the shaft 2, with an end stop 24 to limit movement of the wire 22 (FIG. 10).

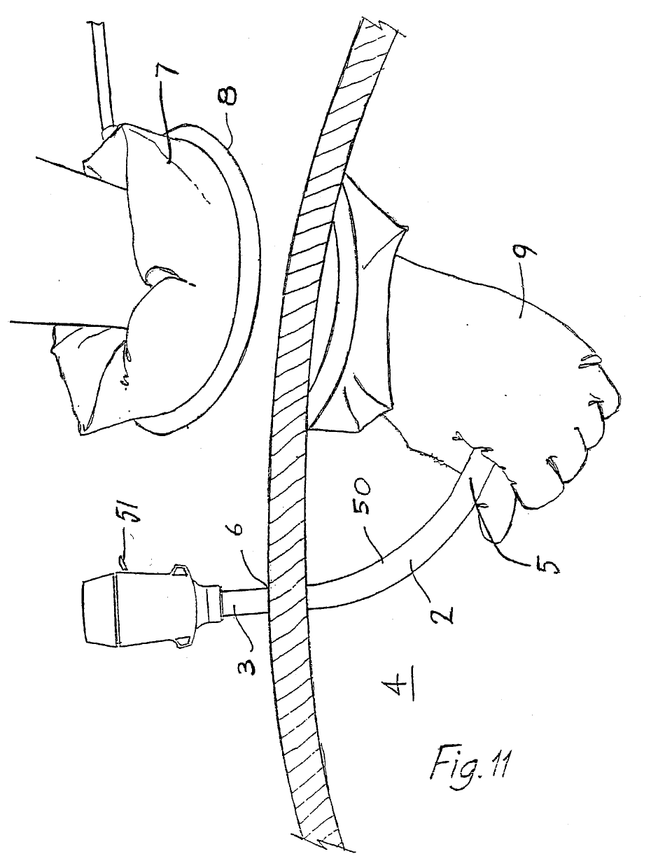

The at least one wire 22 is connected by a clamping arrangement to a locking switch 51 at the proximal end 3 of the shaft 2. After manipulation of the distal end 5 of the shaft 2 into a desired position and/or orientation in the operating space 4 (FIG. 11), the switch 51 is moved from the open position of FIGS. 9 and 11 to the locked position of FIG. 12 to clamp the tensioned wires 22 in place. In this locked position, the position and/or orientation of the distal end 5 of the shaft 2 may not be altered without releasing the lock.

It will be appreciated that the wires 22 may alternatively be positioned along the interior of the shaft 2, or embedded within the shaft 2. It will further be appreciated that a coating, such as a low friction coating or a sleeve may be provided over the wires 22.

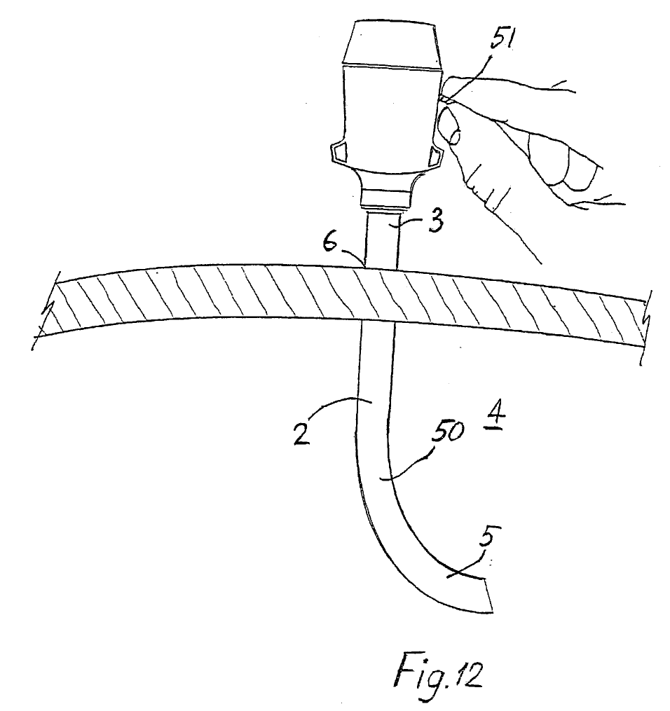

Referring now to FIGS. 13 to 15, there is illustrated another cannula 20 according to the invention, which is similar to the cannula 50 of FIGS. 9 to 12, and similar elements in FIGS. 13 to 15 are assigned the same reference numerals.

In this case, the cannula 20 comprises ancilliary means to facilitate manipulation of the position and/or orientation of the distal end 5 of the shaft 2 from the proximal end 3 of the shaft 2 which is located externally of the operating space 4. The manipulation means comprises, in this case, four malleable wires 22, similar to those described previously with reference to FIG. 10, extending along at least portion of the shaft 2. The wires 22 are slidably received in co-operating guides 23 on the shaft 2, and each wire 22 comprises an end stop 24 to limit movement of the wire 22.

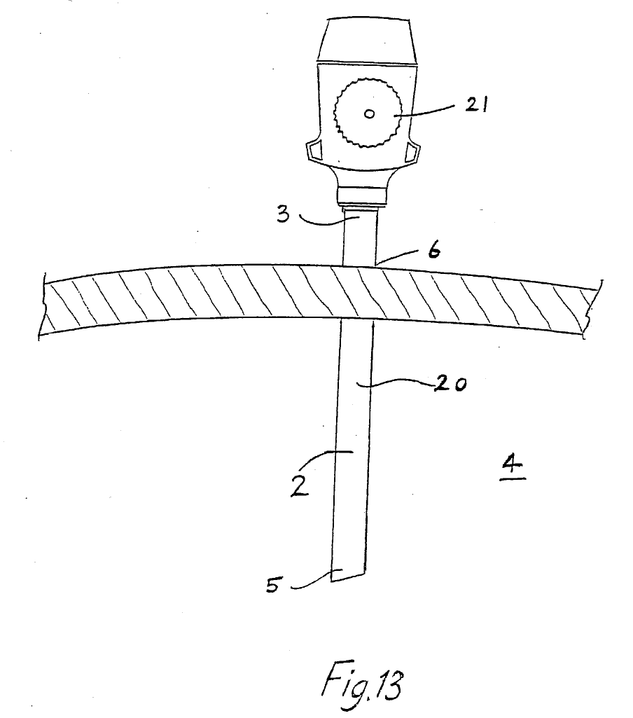

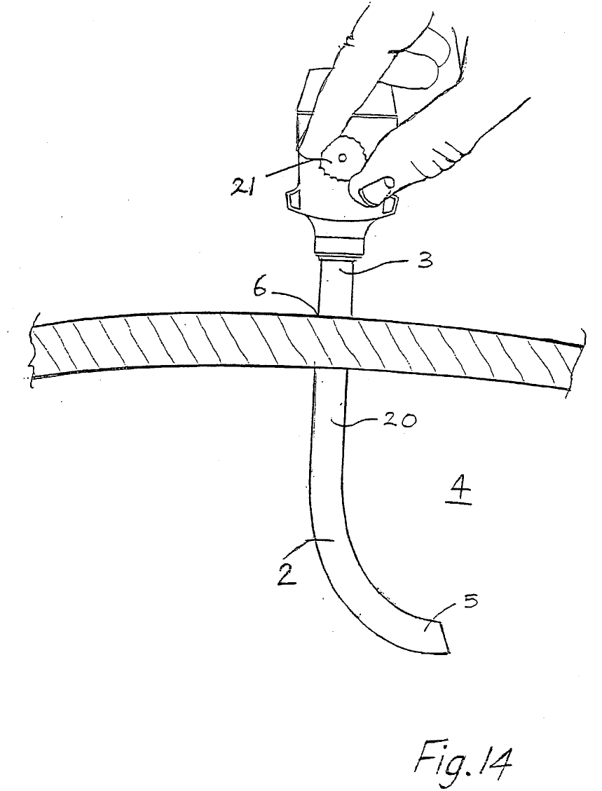

The wires 22 are connected by a clamping arrangement to a control dial 21 at the proximal end 3 of the shaft 2. By turning the dial 21, the wires 22 are pulled relative to one another, and thereby the distal end 5 of the shaft 2 is manipulated into a desired position and/or orientation in the operating space 4 (FIG. 14). After manipulation, the distal end 5 of the shaft 2 maintains the manipulated position and/or orientation due to the malleable nature of the shaft 2, and also due to the malleable nature of the wires 22 (FIG. 15).

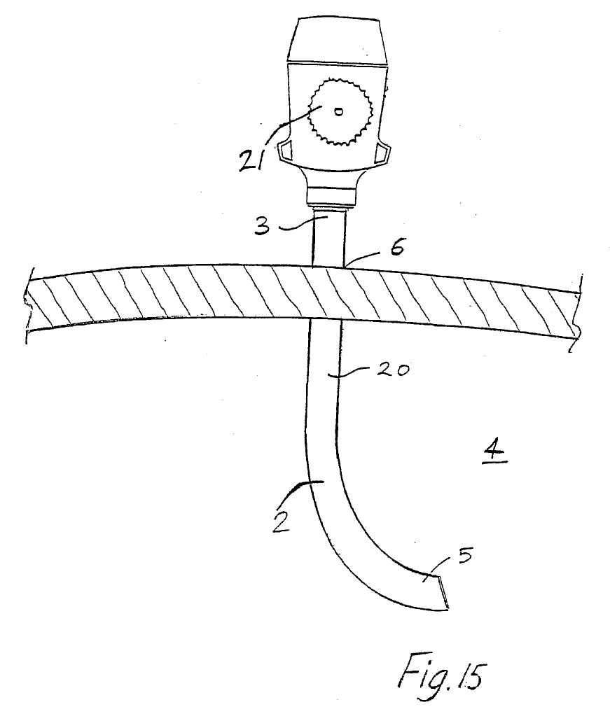

Referring to FIGS. 16 to 18, there is illustrated another cannula 30 according to the invention, which is similar to the cannula 1 of FIGS. 1 to 8, and similar elements in FIGS. 16 to 18 are assigned the same reference numerals.

The shaft 2 has a distal portion 31 adjacent the distal end 5 of the shaft 2, and an intermediate portion 32 intermediate the distal end 5 of the shaft 2 and the proximal end 3 of the shaft 2, as illustrated in FIG. 16.

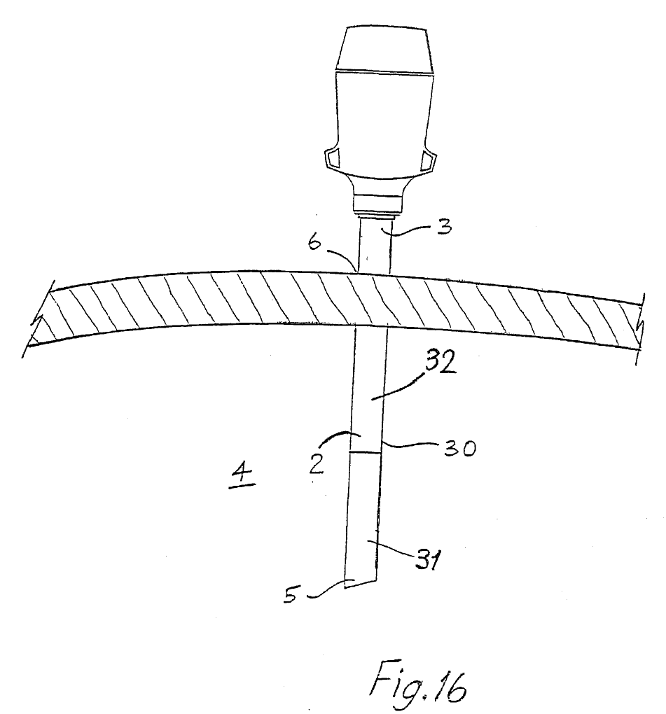

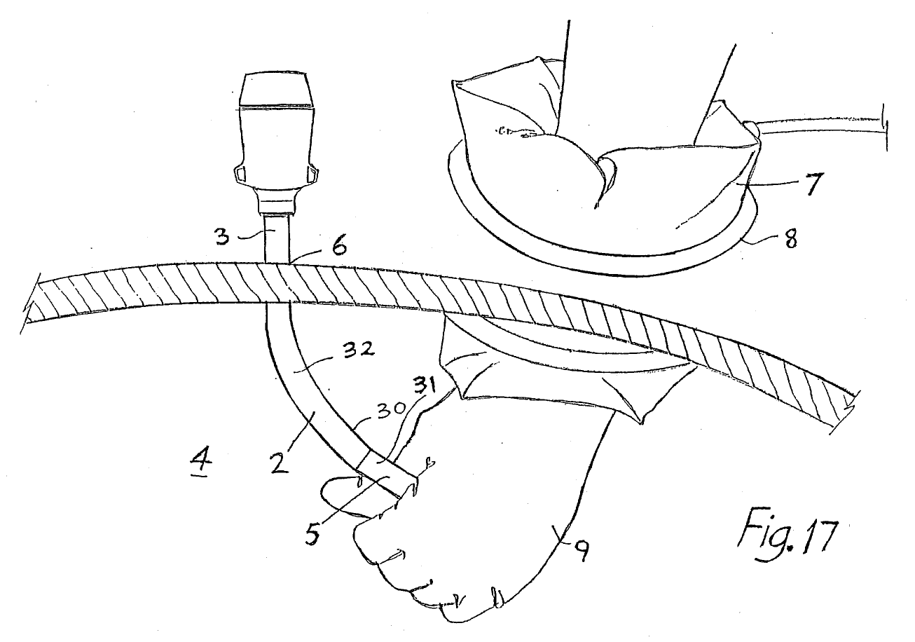

In one case, the distal portion 31 is rigid and the intermediate portion 32 is malleable. The distal portion 31 of the shaft 2 is sufficiently short relative to the intermediate portion 32 of the shaft 2 to enable the distal end 5 of the shaft 2 to be manipulated into a desired position and/or orientation in the operating space 4 by manipulating the malleable intermediate portion 32 of the shaft 2 (FIGS. 17 and 18).

In an alternative case, the distal portion 31 is flexible, and is sufficiently short relative to the malleable intermediate portion 32 of the shaft 2 to facilitate manipulation of the distal end 5 of the shaft 2 into a desired position and/or orientation in the operating space 4.

In another cannula according to the invention, only the distal portion 31 of the shaft 2 is malleable, and the intermediate portion 32 of the shaft 2 is rigid. The distal end 5 of the shaft 2 can be manipulated into a desired position and/or orientation within the operating space by manipulating the malleable distal portion 31 of the shaft 2.

It will be appreciated that for all cannulae of the invention the malleability of the malleable portion of the shaft may vary along the length of the malleable portion of the shaft. For example the shaft 2 may be of gradually increasing malleability moving from the proximal end 3 to the distal end 5 for more fine adjustments at the distal end 5.

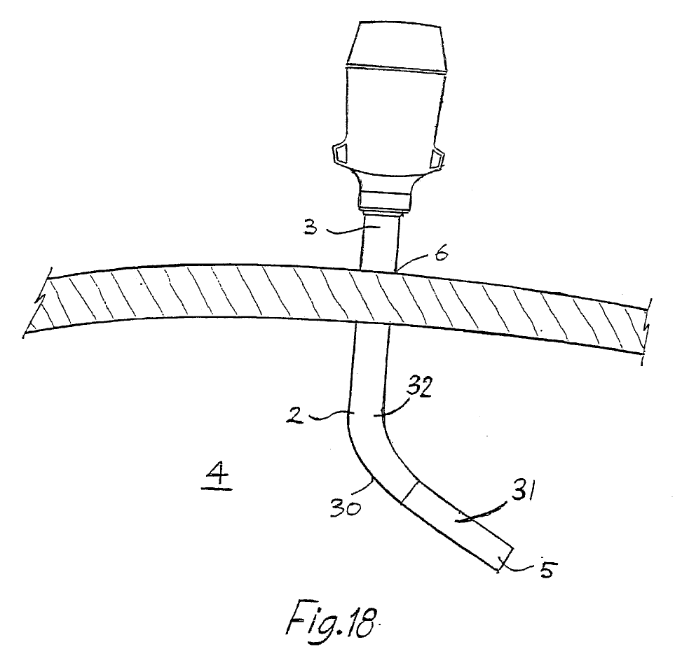

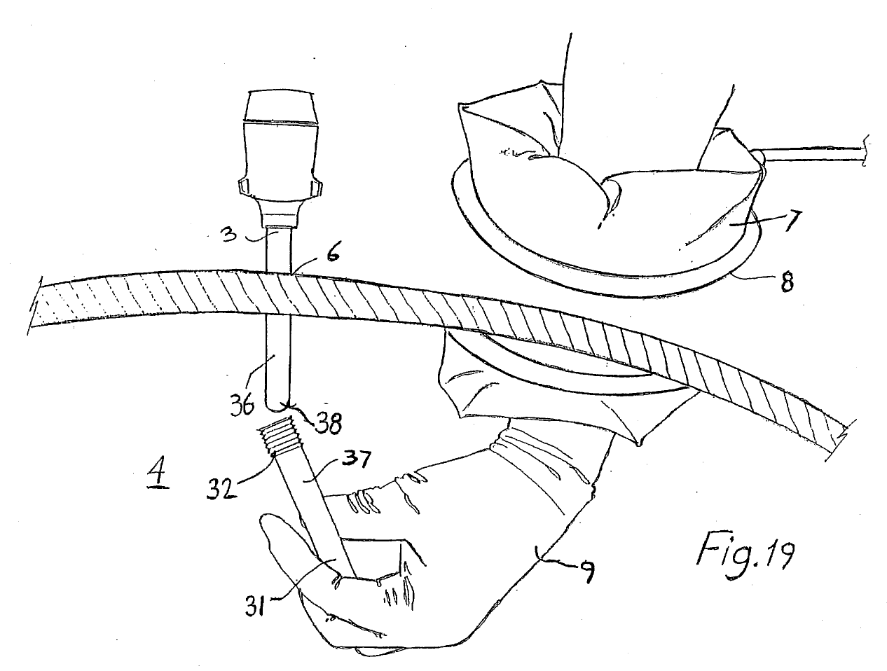

Referring to FIGS. 19 to 21, there is illustrated another cannula 35 according to the invention, which is similar to the cannula 30 of FIGS. 16 to 18, and similar elements in FIGS. 19 to 21 are assigned the same reference numerals.

In this case, the cannula 35 comprises a main shaft body 36 and a shaft extension 37 mounted to the main shaft body 36, preferably in a releasable manner.

The shaft extension 3 7 may be mounted to a distal end 38 of the main shaft body 36 within the operating space 4, as illustrated in FIG. 19, or alternatively externally of the operating space 4.

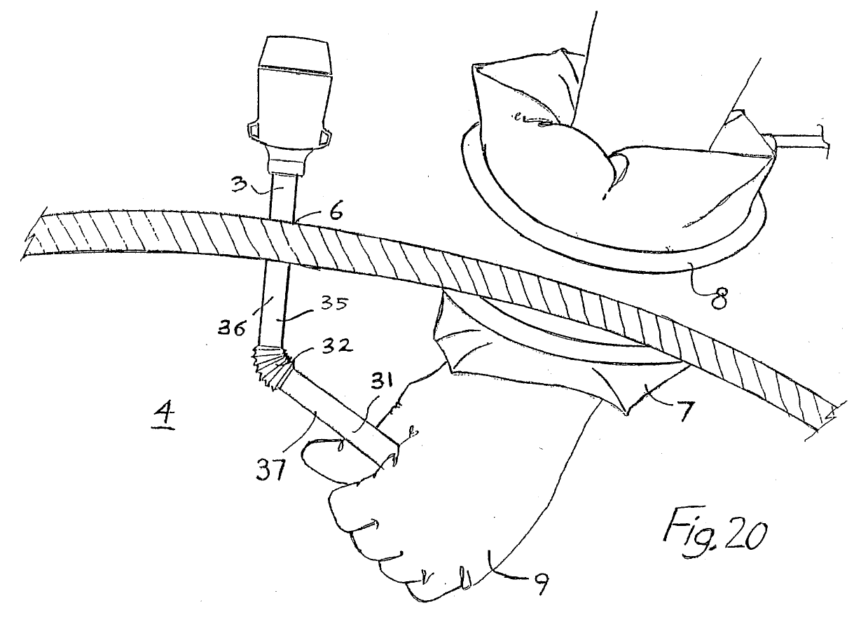

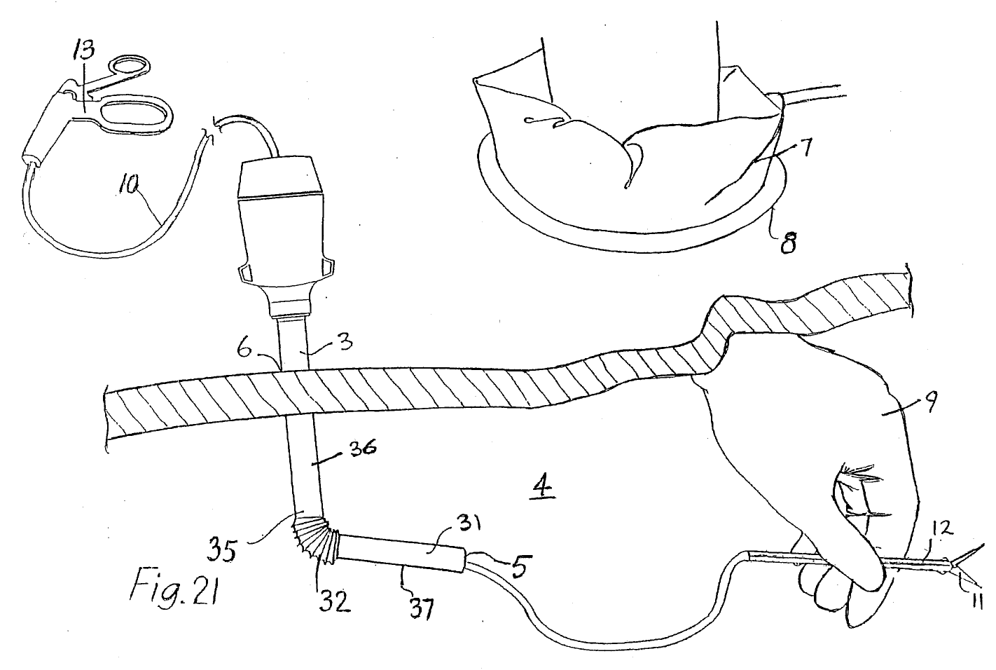

The malleable intermediate portion 32 of the shaft 2 facilitates manipulation of the rigid distal portion 31 of the shaft 2 and thereby the distal end 5 of the shaft 2 into a desired position and/or orientation in the operating space 4 (FIG. 20). A surgical instrument 10 may be partially inserted through the cannula 35 to access a desired position and/or at a desired orientation in the operating space 4 (FIG. 21).

The shaft extension 37 enables the surgeon to selectively convert a standard rigid cannula into an at least partially malleable and/or flexible cannula, as desired.

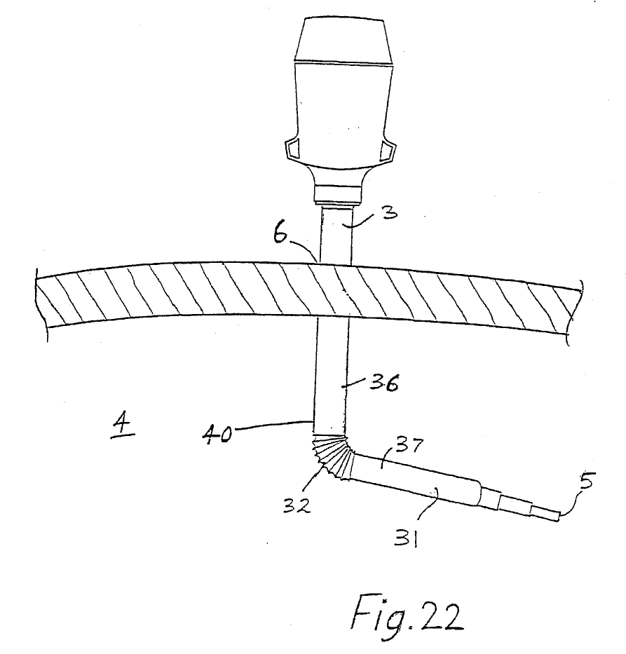

FIGS. 22 and 23 illustrate another cannula 40 according to the invention, which is similar to the cannula 35 of FIGS. 19 to 21, and similar elements in FIGS. 22 and 23 are assigned the same reference numerals.

At least portion of the shaft 2, in this case the rigid distal portion 31 of the shaft 2, is extendable from a retracted configuration (FIG. 22) to an extended configuration (FIG. 23).

The extendable nature of the shaft 2 facilitates enhanced access by a surgical instrument 10 partially inserted through the cannula 40 to a desired position and/or at a desired orientation in the operating space 4 (FIG. 23).

As illustrated in FIGS. 22 and 23, the shaft 2 extends in a telescopic manner, however it will be appreciated that the shaft 2 may alternatively extend in a concertina manner, or in any other suitable manner.

It will further be appreciated that any suitable portion of the shaft 2 may be extendable to provide enhanced accessibility to a desired position in the operating space 4 for a surgical instrument 10 partially inserted through the cannula 40.

It will be understood that other medical devices may be inserted partially or completely through the cannula of the invention to access a desired position and/or at a desired orientation in the operating space.

FIG. 24 illustrates an endoscope 45 partially inserted through the cannula 35, described previously with reference to FIGS. 19 and 20, into the operating space 4. A light source 46 and/or a viewing means 47, such as a television camera, may be passed through lumena in the endoscope 45 (FIG. 24).

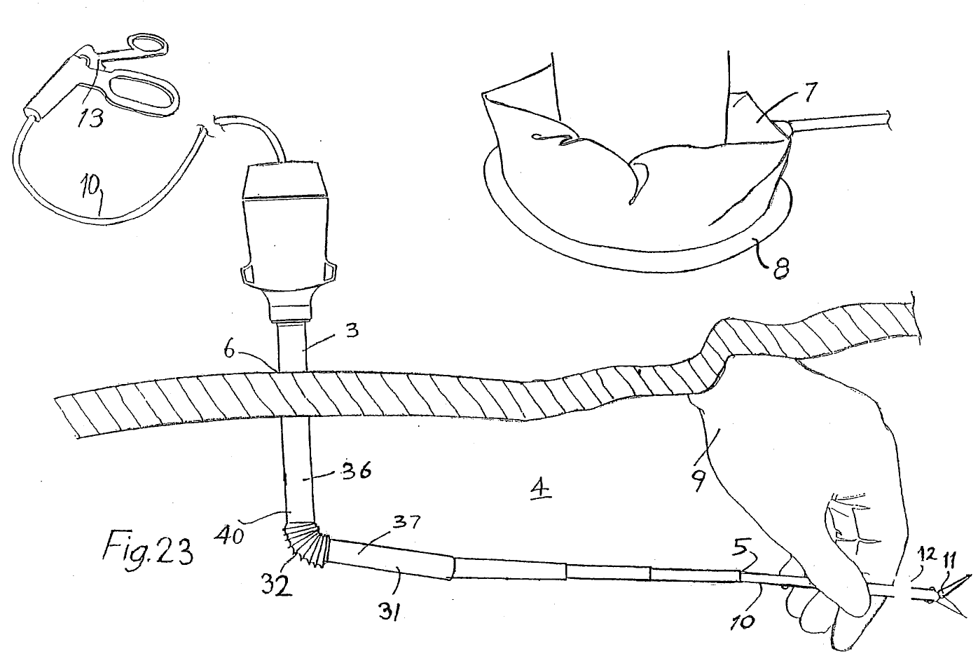

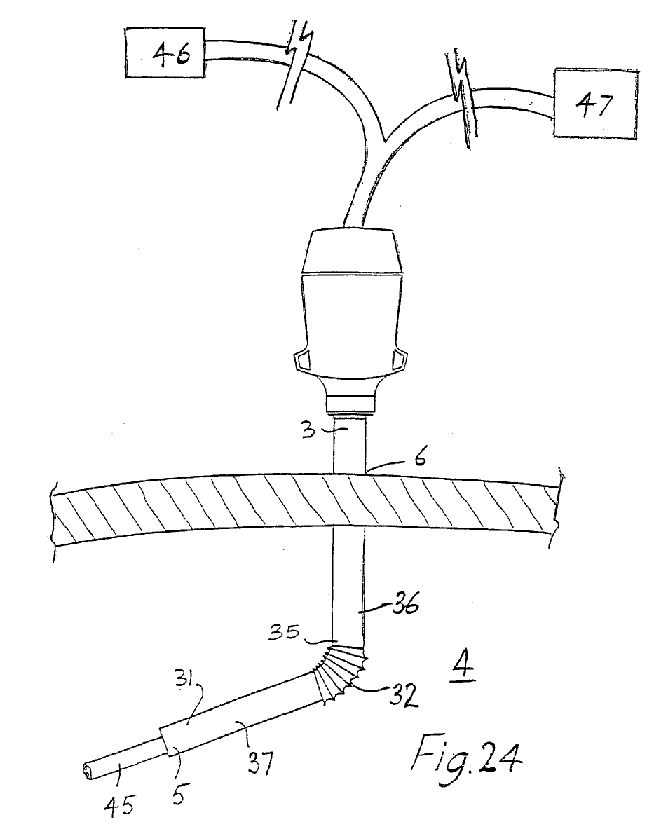

A typical construction for the malleable shaft 2 of the cannula is illustrated in FIGS. 25 and 26. The shaft 2 comprises an inner spring coil 122a, an intermediate spring coil 122b with a greater pitch than the inner spring coil 122a, and an outer shrink-wrapped tube 122c.

The outer tube 122c acts as a sealing jacket around the spring coils 122a, 122b, and in use seals the cannula to the edges of an incision opening. This sealing effect is particularly important when the instrument 1 is used during laparoscopy to prevent insufflation gas from escaping between the cannula and the edges of an incision opening. In this case pneumoperitoneum within the operating space is maintained even during manipulation of the cannula shaft.

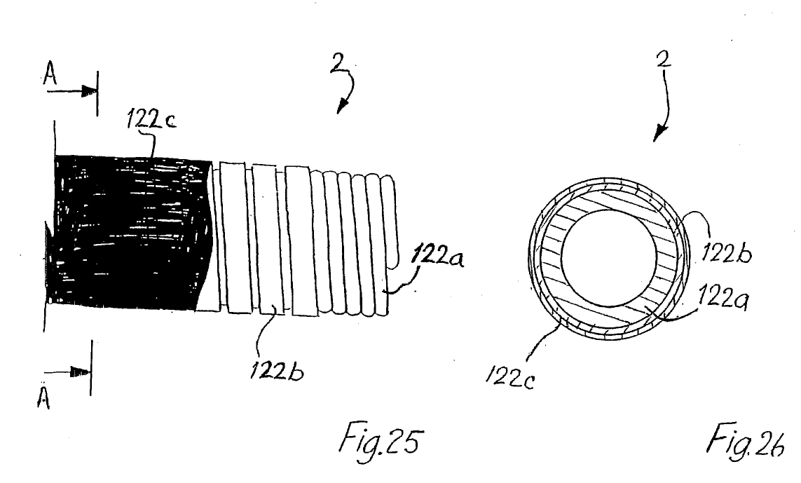

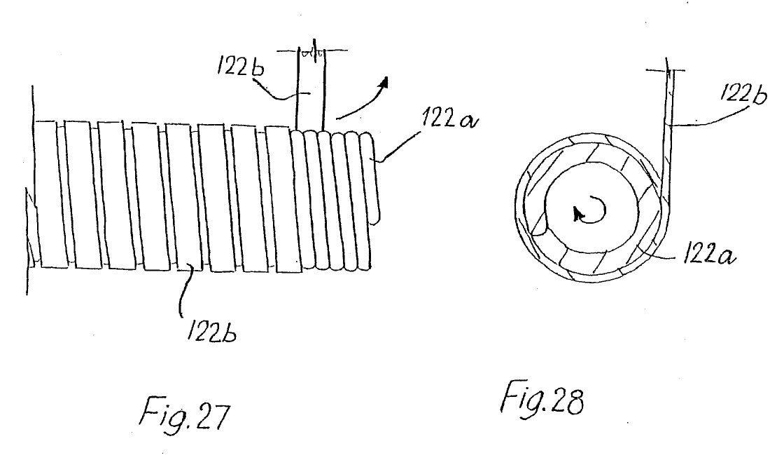

To form the shaft 2, the intermediate spring coil 122b is wrapped around the inner spring coil 122a, as illustrated in FIGS. 27 and 28, and the outer tube 122c is shrink-wrapped around the spring coils 122a, 122b.

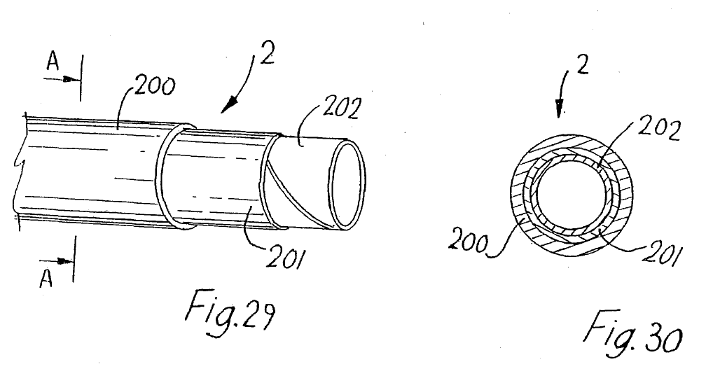

An alternative construction for the shaft 2 of the cannula is illustrated in FIGS. 29 and 30. The shaft 2, in this case, comprises an inner flexible tube 202, typically of a material such as polyvinylchloride (PVC), an intermediate tube 201, typically of a metal such as aluminium, and an outer malleable tube 200, typically of a material such as PVC. The PVC tubing 200, 202 help to prevent kinking in the aluminium tube 201.

The malleable cannulae of the invention as hereinbefore described with reference to the drawings are particularly applicable to use during hand assisted laparoscopic surgery.

However, it will be appreciated that the cannulae provided by the invention are also applicable to other forms of laparoscopic surgery.

One cannula of the invention, suitable for these other forms of laparoscopy, comprises a flexible shaft with means to lock the shaft in a desired manipulated position and/or orientation within the operating space. The flexible cannula may also be steered into the desired position and/or at the desired orientation from a point externally of the operating space.

Another cannula of the invention comprises a non-straight shaft to access a desired position and/or at a desired orientation within the operating space by passing a laparoscopic instrument partially through the non-straight cannula.

The invention is not limited to the embodiments hereinbefore described, with reference to the accompanying drawings, which may be varied in construction and detail.

1. A cannula comprising a shaft extending between a proximal end for location externally of an operating space and a distal end for insertion into the operating space, at least portion of the shaft being malleable to maintain the distal end of the shaft in a manipulated position and/or orientation.

2. A cannula as claimed in claim 1 wherein the shaft is malleable substantially along the length thereof.

3. A cannula as claimed in claim 1 wherein the shaft has a distal portion adjacent the distal end of the shaft, and the distal portion of the shaft is malleable.

4. A cannula as claimed in claim 3 wherein the shaft has an intermediate portion intermediate the distal end of the shaft and the proximal end of the shaft, and the intermediate portion of the shaft is rigid.

5. A cannula as claimed in claim 1 wherein the shaft has an intermediate portion intermediate the distal end of the shaft and the proximal end of the shaft, and the intermediate portion of the shaft is malleable.

6. A cannula as claimed in claim 5 wherein the shaft has a distal portion adjacent the distal end of the shaft, and the distal portion of the shaft is rigid.

7. A cannula as claimed in claim 5 wherein the shaft has a distal portion adjacent the distal end of the shaft, and the distal portion of the shaft is flexible.

8. A cannula as claimed in claim 6 or 7 wherein the distal portion of the shaft is short relative to the intermediate portion of the shaft.

9. A cannula as claimed in any preceding claim wherein the malleability of the malleable portion of the shaft varies along the length of the malleable portion of the shaft.

10. A cannula as claimed in any preceding claim wherein the shaft comprises a main shaft body and a shaft extension mounted to the main shaft body.

11. A cannula as claimed in claim 10 wherein the shaft extension is releasably mounted to the main shaft body.

12. A cannula as claimed in claim 10 or 11 wherein the shaft extension is mounted to a distal end of the main shaft body.

13. A cannula as claimed in any preceding claim wherein at least portion of the shaft is extendable from a retracted configuration to an extended configuration.

14. A cannula as claimed in claim 13 wherein the shaft is telescopically extendable.

15. A cannula as claimed in claim 13 wherein the shaft is extendable in a concertina manner.

16. A cannula as claimed in any preceding claim wherein the cannula comprises an adjuster to facilitate manipulation of the position and/or orientation of the distal end of the shaft from the proximal end of the shaft.

17. A cannula as claimed in any preceding claim wherein the cannula comprises a lock for locking the distal end of the shaft in a desired manipulated position and/or orientation.

18. A cannula as claimed in claim 16 or 17 wherein the adjustor and/or the lock is provided by at least one wire extending along at least portion of the shaft.

19. A cannula as claimed in claim 18 wherein the wire is at least partially malleable.

20. A cannula as claimed in claim 18 or 19 wherein the wire is slidably received in at least one guide on the shaft.

21. A cannula as claimed in any of claims 18 to 20 wherein the wire comprises a stop to limit movement of the wire.

22. A cannula as claimed in any preceding claim wherein at least the malleable portion of the shaft is of a layered construction.

23. A cannula as claimed in claim 22 wherein at least one layer comprises a seal.

24. A cannula as claimed in claim 22 or 23 wherein at least one layer comprises a spring coil.

25. A cannula as claimed in any of claims 22 to 24 wherein at least one layer is of a polymeric material.

26. A cannula as claimed in claim 25 wherein the polymeric material is polyvinylchloride.

27. A cannula as claimed in any of claims 22 to 26 wherein at least one layer is of a metallic material.

28. A cannula as claimed in claim 27 wherein the metallic material is aluminium.

29. A cannula as claimed in any preceding claim wherein the radial dimension of the shaft is in the range of from 2 mm to 15 mm.

30. A cannula as claimed in any preceding claim wherein the cannula is a laparoscopic cannula.

31. A surgical apparatus comprising a cannula as claimed in any preceding claim and a surgical instrument for partial insertion through the cannula.

32. An apparatus as claimed in claim 31 wherein the instrument has a distal portion adjacent a distal end of the instrument, and at least the distal portion of the instrument is flexible.

33. A method of performing minimally invasive surgery, the method comprising the steps of:—
providing a cannula, the cannula having a proximal end and a distal end, and at least portion of the cannula being malleable;
partially inserting the cannula through an opening into an operating space so that the proximal end is located externally of the operating space and the distal end is located within the operating space; and
manipulating the distal end of the cannula into a desired position and/or orientation, the manipulated position and/or orientation being maintained by the malleable nature of the cannula.

34. A method as claimed in claim 33 wherein the cannula is at least partially manipulated before insertion of the cannula into the operating space.

35. A method as claimed in claim 33 or 34 wherein the cannula is at least partially manipulated during insertion of the cannula into the operating space.

36. A method as claimed in claim 35 wherein the cannula is at least partially manipulated by levering the proximal end of the cannula about the opening to the operating space when the cannula is partially inserted through the opening to the operating space.

37. A method as claimed in any of claims 33 to 36 wherein the cannula is at least partially manipulated after insertion of the cannula into the operating space.

38. A method as claimed in any of claims 33 to 37 wherein the method comprises the steps of:—
providing a surgical instrument; and
partially inserting the surgical instrument through the cannula into the operating space.

39. A method as claimed in claim 38 wherein the surgical instrument is inserted through the cannula after the distal end of the cannula has been manipulated into the desired position and/or orientation.

40. A method as claimed in any of claims 33 to 39 wherein the method comprises the step of inserting a hand into the operating space.

41. A method as claimed in claim 40 wherein the method comprises the steps of:—
providing a hand-access device;
mounting the hand-access device at an opening to the operating space; and
inserting the hand through the hand access device into the operating space.

42. A method as claimed in claim 40 or 41 wherein the cannula is at least partially manipulated into the desired position and/or orientation by the hand from within the operating space.

43. A method as claimed in any of claims 33 to 42 wherein the cannula is at least partially manipulated into the desired position and/or orientation from the proximal end of the cannula externally of the operating space.

44. A method as claimed in any of claims 33 to 43 wherein the method comprises the step of locking the distal end of the cannula in the manipulated position and/or orientation.

45. A method of performing minimally invasive abdominal surgery as claimed in any of claims 33 to 44 wherein the operating space is an abdominal cavity.

46. A method as claimed in any of claims 33 to 45 wherein the method comprises the step of extending the cannula from a retracted configuration to an extended configuration.

47. A method as claimed in any of claims 33 to 46 wherein the method comprises the step of mounting an extension to the cannula.

48. A method of performing minimally invasive surgery substantially as hereinbefore described with reference to the accompanying drawings.

49. A cannula comprising a shaft extending between a proximal end for location externally of an operating space and a distal end for insertion into the operating space, at least portion of the shaft being flexible to manipulate the shaft into a desired position and/or orientation, and a lock for locking the shaft in the manipulated position and/or orientation.

50. A cannula comprising a shaft extending between a proximal end for location externally of an operating space and a distal end for insertion into the operating space, at least portion of the shaft being non-straight to access a desired position and/or at a desired orientation within the operating space.

51. A cannula substantially as hereinbefore described with reference to the accompanying drawings.
